(19) United States
(12) Patent Application Publication
(10) Pub. No.: US 2021/0378261 A1
(43) Pub. Date: Dec. 9, 2021

(54) NOVEL GRAS PROBIOTIC BACTERIAL STRAIN TO INHIBIT ACIDOSIS AND LIVER ABSCESSES IN CATTLE

(71) Applicant: NUtech Ventures, Lincoln, NE (US)

(72) Inventor: Samodha Fernando, Lincoln, NE (US)

(21) Appl. No.: 17/250,968

(22) PCT Filed: Oct. 2, 2019
(86) PCT No.: PCT/US2019/054290

§ 371 Date: Apr. 2, 2021

Related U.S. Application Data
(60) Provisional application No. 62/740,235, filed on Oct. 2, 2018.

(51) Int. Cl. A23K 10/18; A23K 50/10; A61K 35/742

(52) U.S. Cl. CPC A23K 10/18; A61K 35/742; A23K 50/10

(57) ABSTRACT

The present disclosure provides compositions and methods of using such compositions that reduce the incidence of, duration of, frequency of, or severity of clinical signs associated with or caused by pathogen infection. Representative pathogens include Streptococcus, Fusobacterium, Escherichia, and Arcanobacterium. In general, the composition includes a quantity of at least one Bacillus species.

### CROSS REFERENCE TO RELATED APPLICATIONS

This application claims priority to U.S. Provisional Application No. 62/740,235, filed Oct. 2, 2018, which is hereby incorporated by reference in its entirety.

### BACKGROUND OF THE DISCLOSURE

The field of the disclosure relates generally to the prevention and treatment of pathogenic organisms that are associated with or cause clinical signs or symptoms in animals. Such pathogenic organisms include Streptococcus bovis, Fusobacterium necrophorum, Escherichia coli, and Arcanobacterium pyogenes. Such pathogens have proven difficult to treat and/or prevent and continue to cause significant health and economic impacts on animal populations. What is needed are compositions that are able to be administered to animals using a variety of different administration modes designed for different infection locations. What is further needed are such compositions that are recognized as safe for administration to animals.

### BRIEF DESCRIPTION OF THE DISCLOSURE

The present disclosure provides compositions comprising at least one probiotic bacterial strain and methods of using such compositions to inhibit growth of pathogenic organisms that are associated with clinical signs in animals. Administration of such compositions can treat existing infections from a variety of different pathogens and reduce the incidence of, duration of, and/or severity of clinical signs or symptoms of infection.

In some forms, the compositions of the disclosure are used to treat an existing infection to reduce the severity, frequency, or duration of clinical signs or symptoms of infection by the pathogen. In some forms, the compositions of the disclosure are used to decrease the incidence, duration, frequency, or severity of clinical signs or symptoms of future infections.

In some forms, the administration is to an animal in need of such administration. In some forms, the animal is susceptible to infection by a pathogen selected from the group consisting of Streptococcus, Fusobacterium, Escherichia, and Arcanobacterium. In some forms, infection by at least one of those pathogens produces clinical signs or symptoms of infection in the animal. In some forms, the animal is human, or non-human. In some forms, the animal is a human, cow, pig, sheep, deer, goat, horse, or chicken.

Preferably the clinical symptoms associated with infection by such pathogens are reduced in frequency, duration, incidence, and/or severity by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or reduced by 100% in comparison to an animal or group of animals that has been subjected to the same challenge conditions but did not receive an administration of the composition. As can be appreciated, such benefits can be achieved during treatment of an active infection, or administration of the composition prior to infection.

In some forms, the compositions include at least one bacterial species. In some forms, the bacterial species synthesize and/or secrete/excrerte bacteriocins or short peptide sequences that inhibit the growth of one or more pathogens. In some forms, the bacterial species is a probiotic species. This probiotic species may be present in the composition as a live bacterial culture, an inactivated or killed bacterial culture, a lyophilized bacterial preparation, a cell culture extract, or any combination thereof. In some forms, the probiotic species is a Bacillus species. In some forms, the Bacillus species is Bacillus licheniformis alone or in combination with Bacillus pumilus. In some forms, the Bacillus licheniformis has a 16S sequence having at least 80, 85, 86, 87, 88, 89, 90, 91, 92, 93, 94, 95, 96, 97, 98, 99, or even 100% sequence homology or sequence identity to any one of SEQ ID NOs. 1-3. In some forms, the Bacillus pumilus has a 16S sequence having at least 80, 85, 86, 87, 88, 89, 90, 91, 92, 93, 94, 95, 96, 97, 98, 99, or even 100% sequence homology or sequence identity to SEQ ID NO. 4.

In some forms, the amount of the probiotic species in the composition comprises In some forms, the amount of probiotic comprises between about 10⁶ to about 10¹² CFU, more preferably 10⁸ to 10¹¹ CFU, and most preferably about 10⁹-10¹⁰ CFU per administration.

In some forms, more than one probiotic species is included in the composition. For example, the composition may include both Bacillus licheniformis and Bacillus pumilus. The probiotic species included in the composition can be at any ratio relative to each other. For example, if there are two species, they may be present in a ratio of 99:1 to 1:99, 90:10 to 10:90, 80:20 to 20:80, 70:30 to 30:70, 60:40 to 40:60, or 50:50. Such combinations work synergistically with one another.

In some forms the pathogen is selected from the group consisting of Streptococcus, Fusobacterium, Escherichia, and Arcanobacterium. In some forms, the Streptococcus pathogen is Streptococcus bovis including ATCC 700410. In some forms, the Fusobacterium pathogen is Fusobacterium necrophorum including ATCC 27852. In some forms, the Escherichia pathogen is Escherichia coli including E. coli 00157. In some forms, the Arcanobacterium is Arcanobacterium pyogenes

Streptococcus bovis (S. bovis) is a species of Gram-positive bacteria that in humans is associated with urinary tract infections, endocarditis, sepsis, and colorectal cancer. S. bovis is a catalase-negative and oxidase-negative, nonmotile, non-sporulating, Gram-positive lactic acid bacterium that grows as pairs or chains of cocci. It is a member of the Lancefield group D streptococci. Most strains are gamma-hemolytic (non-hemolytic), but some also display alpha-hemolytic activity on sheep blood agar plates. S. bovis is a non-enterococci. The S. bovis group includes S. equinus, S. gallolyticus, S. infantarius, and other closely related species; they are the non-enterococcal group D streptococci. Members of this group are esculin positive, 6.5% salt negative, sorbitol negative and produce acetoin. Isolates from the S. bovis group are most frequently encountered in blood cultures from patients with colon cancer. However, S. bovis group organisms (especially S. gallolyticus subsp. gallolyticus and S. infantarius subsp. coli) have been associated with endocarditis. Although infection with S. bovis group organisms occurs with higher frequency in adults than in pediatric patients, these organisms have been reported to cause neonatal sepsis and meningitis S. gallolyticus is commonly found in the alimentary tract of cattle, sheep, and other ruminants and may cause ruminal acidosis or feedlot bloat. It is also associated with spontaneous bacterial peritonitis, a frequent complication occurring in patients affected by cirrhosis. When ruminants consume diets high in starch or sugar, these easily fermentable carbohydrates promote the proliferation of S. bovis in the rumen. Because S. bovis is a lactic acid bacterium, fermentation of these carbohydrates to lactic acid can cause a dramatic decline in ruminal pH, and subsequent development of adverse conditions such as ruminal acidosis or feedlot bloat. Other clinical signs of S. bovis infection include endocarditis, anorexia, weight loss, fatigue, night sweats, foot rot, laminitis, and weakness.

Fusobacterium necrophorum is a Gram-negative, rod-shaped, nonsporeforming, and aerotolerant anaerobe. It is a normal inhabitant of the alimentary, respiratory, and genital tract of animals. It is also a normal inhabitant of the rumen of cattle. The organism is in ruminal contents and adherent to the ruminal wall. Its role in ruminal fermentation is to metabolize lactic acid and degrade feed and epithelial proteins. The ruminal concentration is higher in grain-fed than forage-fed cattle. From the rumen, the organism gains entry into the portal circulation and is trapped in the liver to cause abscesses. The organism is an opportunistic pathogen and a primary causative agent of liver abscesses, an economically important disease of grain-fed cattle. Liver abscesses are often secondary to ruminal acidosis and rumenitis in grain-fed cattle. Two subspecies of F. necrophorum, subsp. necrophorum (biotype A) and subsp. funduliforme (biotype B), are recognized that can be differentiated based on morphological, biochemical, biological and molecular characteristics. The subsp. necrophorum is more virulent and is isolated more frequently from infections than the subsp. funduliforme. Several toxins or secreted products have been implicated as virulence factors. The major factors contributing to ruminal colonization and invasion into the liver are hemagglutinin, endotoxin and leukotoxin, of which leukotoxin is the protective antigen. In some conditions, the organism synergistically interacts with Arcanobacterium pyogenes, a facultative anaerobic organism and a secondary etiologic agent, to cause liver abscesses. The organism is an opportunistic pathogen that causes several necrotic conditions in animals (ie, necrobacillosis), including necrotic laryngitis. Necrotic laryngitis is an acute or chronic F. necrophorum infection of the laryngeal mucosa and cartilage of young cattle, characterized by fever, cough, inspiratory dyspnea, and stridor. It occurs primarily in feedlot cattle 3-18 months of age; however, cases have been documented in calves as young as 5 week and in cattle as old as 24 months. Cases are seen worldwide and year round but appear to be more prevalent in fall and winter. Necrotic laryngitis is most common where cattle are closely confined under unsanitary conditions or in feedlots. The prevalence in feedlot calves is estimated to be 1%-2%. Most cases are sporadic and occur year round, but disease peaks in fall and winter. Mixed upper respiratory tract infections (caused by infectious bovine rhinotracheitis virus and parainfluenza-3 virus; Mycoplasma spp; and bacteria, including Pasteurella and Haemophilus), and the coughing and swallowing associated with these infections, may predispose feedlot cattle to develop laryngeal contact ulcers. These ulcers on the vocal processes and medial angles of arytenoid cartilages are thought to provide a portal of entry for F. necrophorum. In addition to its contribution to liver abscesses, F. necrophorum causes inflammation, necrosis, and edema in the laryngeal mucosa, resulting in variable narrowing of the rima glottidis and inspiratory dyspnea and stridor. If infection extends into the laryngeal cartilage, laryngeal chondritis develops, which may lead to a chronically deformed larynx. Pharyngeal invasion by the organism causes discomfort characterized by painful swallowing motions. Systemic signs of illness have been attributed to the exotoxin produced by F. necrophorum. For clinical findings, a moist, painful cough is often initially noticed. Severe inspiratory dyspnea, characterized by open-mouth breathing with the head and neck extended, and loud inspiratory stridor are common findings. Ptyalism, frequent, painful swallowing motions, bilateral, purulent nasal discharge, and/or a fetid odor to the breath may also be present. Systemic signs may include fever (106° F. [41.1° C.]), anorexia, depression, and hyperemia of the mucous membranes. Untreated calves die in 2-7 days from toxemia and upper airway obstruction. Longterm sequelae include aspiration pneumonia and permanent distortion of the larynx, resulting in a chronic harsh cough and inspiratory dyspnea. Infection is also known to cause or contribute to lesions. Such lesions are typically located over the vocal processes and medial angles of arytenoid cartilages. Acute lesions are characterized by edema and hyperemia surrounding a necrotic ulcer in the laryngeal mucosa; lesions may spread along the vocal folds and processes to involve the cricoarytenoideus dorsalis muscle. In chronic cases, lesions consist of necrotic cartilage associated with a draining tract surrounded by granulation tissue. F. necrophorum is also associated with or the cause of foot rot and laminitis.

Escherichia coli are a large and diverse group of bacteria of the family Enterobacteriaceae commonly found in the lower intestine of warm-blooded organisms like birds and mammals (including cattle and humans). They are Gram-negative, rod-shaped, facultative anaerobe bacteria comprised of literally hundreds of serotypes. A serotype of E. coli is a subgroup within the species that has unique characteristics that distinguish it from other E. coli strains. While these differences are often detectable only at the molecular level, they may result in changes to the physiology or life cycle of the bacteria. New serotypes of E. coli evolve through the natural biological processes of mutation and horizontal gene transfer (the ability to transfer DNA through an existing bacterial population). Most E. coli strains are harmless as part of the normal flora of the gut and can benefit their hosts by aiding in food digestion, producing vitamin K and B-complex vitamins, and by preventing the establishment of harmful bacteria within the intestine. Humans, for instance, need E. coli and other kinds of bacteria within the intestinal tract to remain healthy. E. coli is not always confined to the intestine, and its ability to survive for brief periods outside the body makes it an ideal indicator organism to test for fecal contamination in the environment (water, soil, plants) or in food (vegetables, meat, etc.). Furthermore, serotypes of E. coli are often host-specific, thus making it possible to determine whether the source of fecal contamination originated from birds, humans or other mammals. Although most strains of E. coli are harmless, others can cause illnesses like diarrhea, urinary tract infections, pneumonia and other clinical disease. As a pathogen, E. coli is best known for its ability to cause intestinal disease. Five classes of E. coli that cause diarrheal diseases are currently recognized: enterotoxigenic, enteroinvasive, enterohemorrhagic, enteropathogenic and enteroaggregative. Each class falls within a serological subgroup and has distinct features in how it causes sickness. E. coli O157 is one particular serotype in the enterohemorrhagic category that has become a major concern for cattle producers and their customers because it sometimes causes human illness when introduced into the human intestine (but colonized cattle do not get sick). This infamous strain is more specifically identified as E. coli O157:H7. Microbiologists generally differentiate E. coli strains based on antigens associated with a particular strain, using an “O” and “H” naming system. The “O” designation describes the particular antigen associated with the cell wall of the microbe, while the “H” designation refers to the particular flagella antigen of the cell. This O:H combination is called the serotype. The feature that makes enterohemorrhagic bacteria like E. coli O157 so problematic is the fact that they produce poisons called Shiga toxins that can damage the lining of the human intestine and other tissues. The name “Shiga toxin” is derived from its similarity to a toxin produced by another microbe, Shigella dysenteriae. Apparently, the gene encoding Shiga toxin was transferred from Shigella to E. coli by a virus that infects bacteria (bacteriophage). E. coli bacteria that manufacture these types of toxins are called “Shiga toxin producing E. coli,” or STEC for short (and STEC that cause hemorrhagic colitis and hemolytic uremic syndrome are called enterohemorrhagic E. coli). E. coli O157:H7 is the most commonly identified STEC in North America, and its name is often shortened to E. coli O157, or even just O157. Also, whenever reports of generic E. coli infections or outbreaks are in the news, E. coli O157 is usually the implicated strain. However, many other kinds (serogroups) of STEC can cause disease and are sometimes referred to as “non-O157 STEC” (e.g., serogroups O26, O111 and O103 are the non-O157 STEC that most often cause human illness in the United States). A very important feature for understanding E. coli O157 is the fact that the bacteria do not cause disease in cattle. Thus, E. coli O157 is nonpathogenic for cattle. However, the microbes can be very pathogenic for humans, so ingestion of anything contaminated with E. coli O157 can pose a potential threat to human health. Unfortunately, cattle are common hosts of E. coli O157, along with many other animals. E. coli O157 bacteria do not cause disease in cattle, but healthy cattle can represent a major reservoir for potential human infection. Although various STEC strains have been isolated from a diversity of animal species, the bacteria have been found to be more prevalent in ruminants (more than 435 serotypes of STEC have been recovered from cattle). Furthermore, most human illnesses due to STEC infection have been traced to cattle, their manure or their edible products, especially beef. Since outbreaks of E. coli O157 are often traced back to cattle, every incident where E. coli O157 turns up in the food supply fosters great concern for both the general public and the cattle industry. Research has shown that beef product recalls following outbreaks have a negative effect on demand for beef. For instance, boneless beef prices declined an average of 2.5 percent in the five days following one recall. Therefore, it is in the interest of every cattle producer to employ all available tools that can help reduce the chance of E. coli O157 entering the food supply. Pre- and post-harvest control measures that effectively decrease E. coli O157 in cattle and eliminate contamination of beef products during processing are essential steps toward sustaining a competitive cattle industry.

Arcanobacterium pyogenes is a species of bacteria that are nonmotile, facultatively anaerobic, and Gram-positive. The cells typically measure 0.5 by 2.0 μm. They appear as pleomorphic or coccoid rods. They tend to be grouped singly, or in short chains. Sometimes, they are grouped into V-shaped pairs. The species was originally classified as Corynebacterium pyogenes, then Actinomyces pyogenes, then Arcanobacterium pyogenes, and is now officially classified as Trueperella pyogenes (T. pyogenes). All such names refer to the same organism and are used interchangeably herein. The specific name pyogenes is used in various bacterial genera and was derived from the Greek word puon or Latin word pyum, and the suffix -genes, yielding pyogenes, meaning “pus-producing”. T. pyogenes is found in the urogenital, gastrointestinal, and upper respiratory tracts of cattle, goats, horses, musk deer, pigs, and sheep, in which it may cause abscesses, mastitis, metritis, and pneumonia. It can thrive in either anaerobic or aerobic environments, but is ideally suited to one with high (about 7%) levels of carbon dioxide. Although it can infect a wide variety of tissues, Trueperella pyogenes is the most common cause of “summer mastitis” in cattle, and the most common cause of pyometra in dogs. It is also commonly associated with suppurative diseases in cattle, including abscesses, chronic endometritis and embolic pneumonia, and bovine mastitis. Overseas, A. pyogenes mastitis is widespread and usually sporadic. The source of infection is considered to be endogenous, as A. pyogenes can be recovered from the mucous membranes, skin, tonsils and genital tract of clinically normal cattle. Infection usually follows localized tissue damage from trauma, foreign body penetration or another infectious process.

The term “administering” as used herein includes all means of introducing the live, inactivated, or killed bacterial strains and/or their extracts and/or their lyophilized preparations to a subject, including, but not limited to, oral (po), inhalation, buccal, sublingual, via suppository, microbiome transplantation, topical, dermal, and the like. The strains and/or extracts and/or preparations described herein may be administered in unit dosage forms and/or formulations containing conventional nontoxic pharmaceutically-acceptable carriers, adjuvants, and vehicles.

Illustrative formats for oral administration include liquids, solids, tablets, pills, capsules, solids in a liquid medium, powders, lozenges, straws, sachets, cachets, solutions, elixirs, suspensions, emulsions, solutions, syrups, sprays, aerosols, soft and hard gelatin capsules, sterile packaged powders, or combined with or introduced into a food product as a direct fed microbial (such as in a top dressing of animal feed).

In particularly suitable embodiments, the methods of the present disclosure include incorporating the probiotic(s) or extracts or lyophilized preparations thereof into the diet of the subject. In some forms, the probiotics can include live cultures, inactivated or dead cultures, cell culture extracts, or lyophilized preparations.

In some forms, the compositions may be included in an ointment or spray for topical administration.

In some forms, the compositions may be included or incorporated as a top dressing on animal feed.

In some forms, the compositions may be included in paste, such as toothpaste, an oral rinse or spray, or liquid for different oral administration methods.

In some embodiments, a therapeutically effective amount of the probiotic(s) or extracts or lyophilized preparations thereof in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, for example, Bacillus licheniformis alone or in combination with Bacillus pumilus and any extract or lyophilized preparation thereof can be administered in the form of liquids, solids, tablets, pills, capsules, solids in a liquid medium, powders, lozenges, straws, sachets, cachets, solutions, elixirs, suspensions, emulsions, solutions, syrups, pastes, ointments, sprays including aerosols (as a solid or in a liquid medium), soft and hard gelatin capsules, sterile packaged powders, or combined with or introduced into a food product as a direct fed microbial.

The term “therapeutically effective amount” as used herein, refers to that amount of active compound or agent that elicits the biological or medicinal response in a tissue system, animal or human that is being sought by a researcher, veterinarian, medical doctor or other clinician, which includes alleviation of the symptoms of the disease, condition or ailment being treated. In one aspect, the therapeutically effective amount is that which may treat or alleviate the condition, disease or symptoms of the disease at a reasonable benefit/risk ratio applicable to any medical treatment. The specific therapeutically-effective dose level for any particular patient will depend upon a variety of factors, including the severity of the condition being treated; the specific composition employed; the age, body weight, general health, gender and diet of the patient: the time of administration, route of administration, and the duration of the treatment; drugs used in combination or coincidentally with the probiotic(s) or extracts thereof and prebiotic(s); and like factors well known to the medical doctor, researcher, veterinarian, or other clinician of ordinary skill.

It is also appreciated that the therapeutically effective amount is advantageously selected with reference to any toxicity, or other undesirable side effect, that might occur during administration of the probiotic(s) such as Bacillus licheniformis, and any extract or preparation thereof, as well as any combination thereof.

“Prebiotic” as used herein, refers to a substrate that exerts health benefits, which may include, but are not limited to, selective stimulation of the growth and/or activity of one or a limited number of beneficial gut bacteria, stimulation of the growth and/or activity of ingested probiotic microorganisms, selective reduction in gut pathogens, and favorable influence on gut short chain fatty acid profile. Some combinations of probiotic(s) such as Bacillus licheniformis alone or in combination with Bacillus pumilus and any extract or combination thereof and/or at least one prebiotic will act synergistically with one another. Such prebiotics may be naturally-occurring, synthetic, or developed through the genetic manipulation of organisms and/or plants, whether such new source is now known or developed later. Prebiotics useful in the present disclosure may include soluble starch, yeast extract, oligosaccharides, polysaccharides, and other prebiotics that contain fructose, xylose, soya, galactose, glucose and mannose.

More specifically, prebiotics useful in the present disclosure may include soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, and gentio-oligosaccharides.

In an embodiment, the total amount of prebiotics present in the composition may be from about 1.0 g/L to about 30.0 g/L including 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 g/L of the composition. More preferably, the total amount of prebiotics present in the nutritional composition may be from about 2.0 g/L and about 8.0 g/L of the composition. In some embodiments, the total amount of prebiotics present in the nutritional composition may be from about 0.1 g/100 kcal to about 1 g/100 kcal. In certain embodiments, the total amount of prebiotics present in the nutritional composition may be from about 0.3 g/100 kcal to about 0.7 g/100 kcal.

The prebiotics of this disclosure can be in the same formulation as the probiotics, or in a separate dosage form. In some forms, the prebiotic and probiotic are in contact with one another in the composition. The prebiotics and compositions of this disclosure may also be taken with carbohydrate or fiber to increase their effectiveness.

The compositions of the present disclosure can also include one or more additional active ingredients, excipients, dissolution agents, surfactants, antioxidants, antiseptics, preservatives, penetrants, osmoprotectants, cryoprotectants, and combinations thereof.

Various excipients can be mixed with the compositions as would be known to those skilled in the art. Suitable excipients include, for example, microcrystalline cellulose, maltodextrin, colloidal silicon dioxide, lactose, starch, sorbitol, cyclodextrin and combinations thereof.

Suitable dissolution agents include, for example, organic acids such as citric acid, fumaric acid, lactic acid, tartaric acid, succinic acid, ascorbic acid, acetic acid, malic acid, glutaric acid and adipic acid, and can be used alone or in combination. These agents can also be combined with salts of the acids, e.g. sodium citrate with citric acid, to produce a buffer system.

Suitable surfactants include, for example, sodium lauryl sulphate, polyethylene separates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxamers, polyvinyl alcohol and sorbitan fatty acid esters.

Suitable antioxidants include, for example, sodium metabisulfite, tocopherols such as α, β, δ-tocopherol esters and α-tocopherol acetate, ascorbic acid and pharmaceutically acceptable salts thereof, ascorbyl palmitate, alkyl gallates (e.g., propyl gallate, TENOX® PG, TENOX® S-1), sulfites and pharmaceutically acceptable salts thereof, butylated hydroxyanisole, butylated hydroxytoluene, and monothioglycerol.

Suitable antiseptics include, for example, chlorhexidine gluconate, glucono delta-lactone, methylparaben, sodium hydroxide, and combinations thereof.

Suitable preservatives include parabens. Suitable parabens include, for example, methylparaben (E number E218), ethylparaben (E214), propylparaben (E216), butylparaben and heptylparaben (E209). Less common parabens include isobutylparaben, isopropylparaben, benzylparaben and their sodium salts.

Suitable penetrants include, for example, sulphoxides (e.g., dimethyl sulphoxide, dimethylacetamide, dimethylformamide), azone (1-dodecylazacycloheptan-2-one or laurocapran), pyrrolidones (e.g., N-methyl-2-pyrolidone), fatty acids (e.g., oleic acid, lauric acid, myristic acid, capric acid), essential oils (e.g., Eucalyptus, Chenopodium, ylang-ylang, L-menthol), terpenes (e.g., sesquiterpene), terpenoids, oxazolidinones (e.g., 4-decyloxazolidin-2-one), and urea.

Lyophilization of the probiotic species can be done using any conventional methodology. For example, one method of producing a lyophilized form of a probiotic for use with the present disclosure generally comprises the following protocol: the probiotic strain(s) are centrifuged at 3000×g for 15 minutes at room temperature and the resulting cell pellet is suspended in 10 ml of 20% glycerol in spent media resuspension solution (the media collected after centrifugation is mixed with 50% sterile glycerol to generate a 20% resuspension solution). The resulting cell suspension is snap frozen in liquid nitrogen and is then freeze dried to obtain a freeze dried viable cell product. 10 milligrams of the freeze dried cells is suspended in peptone water and is spread on brain heart infusion agar plates to determine viable colony forming units (CFUs) per milligram of freeze dried product. Alternatively, lyophilization can be scaled up in an appropriate industrial or commercial manufacturing processes wherein cells are harvested from high-cell density fermentors by continuous centrifugation and the slurries are frozen and lyophilized.

An “immunogenic composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

“Incidence” in clinical signs or symptoms can refer to either the overall number of clinical signs or symptoms present in an individual animal or can refer to the relative number of animals in a group of animals that exhibit clinical signs or symptoms.

Additionally, the composition can include one or more pharmaceutical-acceptable or veterinary-acceptable carriers. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like.

The compositions and methods of the present disclosure can also comprise the addition of any stabilizing agent, such as for example saccharides, trehalose, mannitol, saccharose and the like, to increase and/or maintain product shelf-life and/or to enhance stability.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

In some forms of the disclosure, the composition is in the form of a topical spray or ointment that is applied directly to infected areas of an animal in order to treat current infections including those of the pathogens described herein. For example, the composition may be in the form of an ointment that is applied to the hooves of cattle in order to treat or prevent foot rot. In other forms, it may be in the form of a spray or dip to treat or prevent mastitis. In still other forms, it may be in the form of a paste or oral rinse to treat or prevent mouth and throat related infections. In other forms of the disclosure, the spray or dip is administered to carcasses, hides, meat products in order to prevent the growth of pathogens prior to or during processing. In still other forms, the composition is applied to a food product or fed directly to the animal as a direct fed microbial.

Administration of compositions of the disclosure can occur once, or can occur multiple times as appropriate.

### BRIEF DESCRIPTION OF THE DRAWINGS

FIG. 1A is a photograph illustrating the results from initial screening with Fusobacterium necrophorum ATCC 27852;

FIG. 1B is a photograph illustrating the results from initial screening with Streptococcus bovis ATCC 700410;

FIG. 1C is a photograph illustrating the results from a secondary screening of top candidates using Streptococcus bovis ATCC 700410 to further confirm the ability to inhibit Fusobacterium necrophorum ATCC 27852, and identifying that the top isolates, based on 16S rDNA sequencing of the full length gene, were Bacillus licheniformis;

FIG. 1D is a photograph illustrating the results from a secondary screening of top candidates using Fusobacterium necrophorum ATCC 27852 to further confirm the ability to inhibit Streptococcus bovis ATCC 700410, and identifying that the top isolates, based on 16S rDNA sequencing of the full length gene, were Bacillus licheniformis;

FIG. 2A is a photograph illustrating an antimicrobial assay against Streptococcus bovis ATCC 700410 using a cell extract from an overnight culture of Bacillus licheniformis to evaluate inhibition of Streptococcus bovis;

FIG. 2B is a photograph illustrating an antimicrobial assay against Streptococcus bovis ATCC 700410 using lyophilized cells from an overnight culture of Bacillus licheniformis to evaluate inhibition of Streptococcus bovis;

FIG. 2C is a photograph illustrating an antimicrobial assay against Streptococcus bovis ATCC 700410 using heat killed cells from an overnight culture of Bacillus licheniformis to evaluate inhibition of Streptococcus bovis;

FIG. 3A is a photograph illustrating an antimicrobial assay demonstrating inhibition against Escherichia coli O157;

FIG. 3B is a photograph illustrating an antimicrobial assay demonstrating inhibition against Arcanobacterium pyogenes;

FIG. 4 is a graph illustrating the decrease in liver abscesses in animals receiving an administration of a composition of the disclosure in comparison to animals that did not receive such an administration;

FIG. 5 is a chart comparing the frequency of animals having liver abscess in animals that received an administration of a composition of the disclosure and in animals that did not receive such an administration; and

FIG. 6 is a graph illustrating the fold-difference in liver abscess presence in animals that did or did not receive an administration of a composition of the disclosure.

### DETAILED DESCRIPTION OF THE INVENTION

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Example 1

### Methods

### Isolation of the Bacterial Strains

Rumen samples were collected from beef cattle through the rumen fistula using a manual pump from 4 animals. Each animal was on a different diet. Sampling was performed in a manner ensuring the presence of rumen digest particles. The rumen fluid from each animal was mixed together to increase bacterial diversity. Equal amounts (˜10 g) of each rumen sample were mixed together in a 50 ml conical tube and sterile PBS was added to bring the volume to 50 ml. The tube was shaken vigorously for 5 minutes using a vortexer. Aliquots of this sample were incubated at 70° C. for 1 hour to reduce the growth of vegetative cells and to stress the spores in the sample to germinate. The sample was serially diluted (10¹-10⁴) and plated on ISP 2, ISP 4, brain heart infusion (BHI) and casein starch agar (Himedia Laboratories, India) with 50 μg/ml cycloheximide (Sigma Aldrich, St Louis, Mo.) and 20 μg/ml Nalidixic acid (Acros Organics, NJ) using a spiral plater that further dilutes the sample as it is plated. The plates were incubated anaerobically at 37° C. for 14 days.

Screening for Isolates Against Streptococcus bovis and Fusobacterium necrophorum

After incubation of 24 hrs, 48 hrs, 72 hrs and 96 hrs, all separated single colonies were picked into 96 well plates containing brain heart Infusion (BHI) broth (Himedia Laboratories, India) at each time point and were incubated anaerobically and aerobically at 37° C. After the presence of visible growth in the wells, sterile glycerol was added to a final concentration of 25% and the resulting isolates were stored in −80° C. until used for screening experiments.

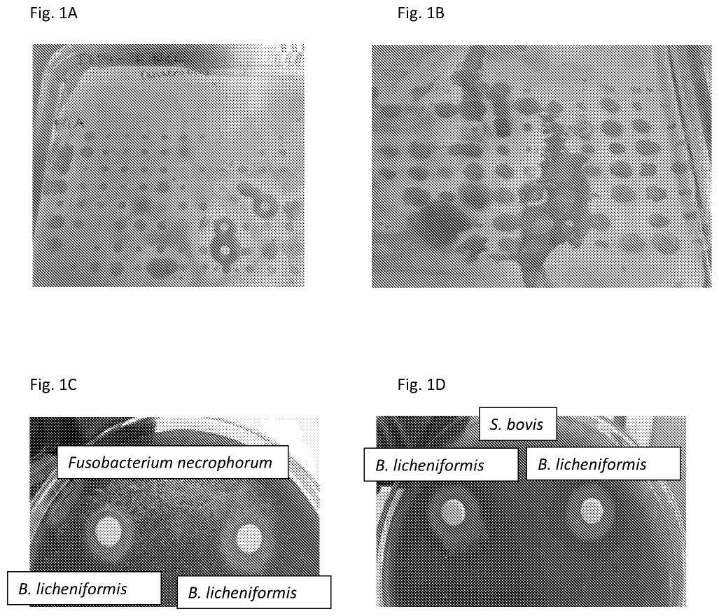

Screening for isolates that are inhibitory towards Streptococcus bovis and Fusobacterium necrophorum was performed as follows in low profile bioassay plates (Thermo Scientific). Briefly, 1000 μl of overnight cultures of Streptococcus bovis (ATCC 700410 (JB1)) and 2-day cultures of Fusobacterium necrophorum (ATCC27852), were independently plated on BHI (BD BBL™, Sparks, Md.) plates and were allowed to dry for 15 min. The dried plates were then spotted with the isolates from the 96 well plates using a plate replicator. The Streptococcus bovis test organism was grown on BHI and Fusobacterium necrophorum was grown on cooked meat medium (Hardy Diagnostics). The plates were incubated anaerobically at 37° C. Plates were checked periodically for clear zones around the isolates, which was indicative of the isolate producing an inhibitory product against the test strains (Streptococcus bovis or Fusobacterium necrophorum). Positive isolates were re-grown from the 96-well plates and glycerol stocks were prepared for archiving, which contained 25% glycerol. Additionally, a secondary screening was performed on the positive strains to confirm inhibition of the test strain. The isolates positive for one test organism were tested against the other test organism to identify isolates with the capacity to inhibit both Streptococcus bovis and Fusobacterium necrophorum. The resulting isolates with the capacity to inhibit the growth of both Streptococcus bovis and Fusobacterium necrophorum were identified for further investigation (see FIGS. 1A-1D).

### Characterization of the Microbial Strains

To characterize the microbial isolates and to confirm the identity of the test strains, 16S rRNA gene was sequenced. Briefly, overnight cultures of each strain was use for DNA extraction using the QuickExtract™—Bacteria DNA extraction kit (Epicenter, Madison, Wis.) according to the manufacturer's protocol. The resulting DNA was used for PCR-based amplification using universal 16S rRNA primers (27F and 1492R). The PCR was done using the Terra™ PCR Direct polymerase mix according to the manufacturers protocol (Clonetech Laboratories, Mountain view, CA). The PCR conditions were, 98° C. for 2 min, followed by 30 cycles of 98° C. for 10 sec, 58° C. for 30 sec, and 68° C. for 90 sec. A final extension of 68° C. for 4 min was used at the end of the 30 cycles. The PCR products were visualized using agarose gel electrophoresis to ensure a single product. The resulting 1500 bp PCR product was purified using shimp alkaline phosphatase and exonuclease and was sequenced from both directions using the 27F and 1492 primers. Sequencing was performed at Eurifins Genomics according to their protocol. The sequencing results generated were matched against the NCBI non-redundant database and the ribosomal database project to identify the taxonomic classification of the unknown isolates and also to verify the taxonomy of the test strains.

Based on the 16S sequencing results, two of the isolates were identified as Bacillus licheniformis UNL07 which is on the US Federal Drug administration's (FDA's) generally accepted as safe (GRAS) list and was identified for further testing.

Whole Genome Sequencing of Bacillus licheniformis UNL07

Overnight cultures of B. licheniformis were used for DNA extraction using the QuickExtract™—Bacteria DNA extraction kit (Epicenter, Madison, Wis.) according to the manufacturer's protocol. The resulting DNA was used for library preparation for whole genome sequencing using the Illumina MiSeq system. Briefly, 600 ng of total DNA was used for Illumina library preparation as described by the manufacturer using the NEBNext Ultra II DNA Library Prep Kit for Illumina (New England Biolabs). Single barcoded adapters were used for the library preparation. The resulting libraries were subjected to size selection (fragment range of 500-1200 bp) using sonication and the Pippin Prep System with 1.5% gel cassettes (Sage Science). The resulting libraries were pooled and sequenced on the Illumina MiSeq sequencing platform using the 250 bp paired-end sequencing strategy to obtain a depth of at least 50× coverage of each genome. Bridge amplification and sequencing will be performed as described by the manufacturer.

Bioinformatic Analysis of the Bacillus licheniformis UNL07 Genome

Sequences generated from MiSeq runs was demultiplexed and processed independently. Briefly, Trimmomatic, version 0.33, was used with a custom adaptor fasta file to trim off adaptor sequences and retain sequences with a minimum length of 130 bp. Artificial duplicates were removed using cd-hit-454. Additionally, a 30 bp sliding window with a quality threshold of 30 was used to trim low quality reads. Ambiguous bases were removed using the ‘fastq_filter’ command in USEARCH.

All resulting reads were assembled with SPAdes. Unpaired reads were also used as input with kmer lengths of 21, 33, 55, 77, 99, and 127, under the ‘single cell setting (--sc)’ within SPAdes. The resulting contigs were further analyzed using KmerFinderJS, which can be found on the internet at the omictools/kmerfinderjs-tool website, to identify bacterial species information based on the whole genome and to identify the similarity to closest genomes in the databases. The results of this analysis is shown in Table 1.

TABLE 1
Bacillus Lichenoformis

| | | Total | Total | | | | |
|---|---|---|---|---|---|---|---|
| | | query | template | | | | |
| | | coverage | coverage | Total | | | |
| Template | p_Value | [%] | [%] | depth | Description | TAXID | Species |
| GCF_0018960 | 1.01E−22 | 87.05 | 96.71 | 0.97 | NZ_CP018249.1 | 1856406 | Bacillus sp. |
| 25.1_ASM189 | | | | | | | H15-1 |
| 602vl | | | | | | | |
| GCF_0032538 | 1.01E−22 | 86.88 | 95.89 | 0.96 | NZ_CP021970.11402 | 1402 | Bacillus licheniformis |
| 15.1_ASM325 | | | | | | | CBA7132 |
| 381vl | | | | | | | |
| GCF_0020740 | 1.01E−04 | 86.66 | 94.11 | 0.94 | NZ_CP014795.1 | 1402 | Bacillus licheniformis |
| 75.1_ASM207 | | | | | | | |
| 407vl | | | | | | | |
| GCF_0022368 | 1.01E−22 | 86.34 | 93.65 | 0.94 | NZ_CP022477.1 | 1402 | Bacillus licheniformis |
| 95.1_ASM223 | | | | | | | |
| 689vl | | | | | | | |
| GCF_0017261 | 1.01E−22 | 85.74 | 91.79 | 0.91 | NZ_CP017247.1 | 1402 | Bacillus licheniformis |
| 25.1_ASM172 | | | | | | | |
| 612vl | | | | | | | |
| GCF_0023932 | 1.01E−22 | 44.02 | 45.68 | 0.46 | NZ_CP023168.1 | 1648923 | Bacillus paralicheniformis |
| 25.1_ASM239 | | | | | | | |
| 322vl | | | | | | | |
| GCF_0018958 | 1.01E−22 | 10.45 | 11.21 | 0.12 | NZ_CP018197.1 | 561879 | Bacillus safensis |
| 85.1_ASM189 | | | | | | | |
| 588vl | | | | | | | |
| GCF_0019386 | 1.01E−22 | 9.82 | 10.55 | 0.11 | NZ_CP015607.1 | 561879 | Bacillus safensis |
| 65.1_ASM193 | | | | | | | |
| 866vl | | | | | | | |
| GCF_0008008 | 1.01E−22 | 9.01 | 10.05 | 0.11 | NZ_CP010075.1 | 756828 | Bacillus sp. |
| 25.1_ASM800 | | | | | | | WP8 |
| 82vl | | | | | | | |
| GCF_9001869 | 1.01E−06 | 6.3 | 6.73 | 0.07 | NZ_LT906438.1 | 1408 | Bacillus pumilus |
| 55.149386E | | | | | | | |
| 02 | | | | | | | |

### Preparing Cell Extracts for Antimicrobial Assays

Based on published studies and previous methods, the cell extracts of the two isolates were purified as described in Berditsch et al. Briefly, 5 ml of the cultures were centrifuged at 3000×g for 15 min at room temperature. The cell pellet was resuspended in the lysis buffer (150 mM NaCl and 20 mM HCL) and was incubated at 80° C. for 15 min, followed by addition of absolute ethanol (1:1 mix—final concentration of 50%). The slurry was then incubated at room temperature with gentle horizontal agitation for 1 hour. Following, extraction with ethanol, the slurry was centrifuged and the supernatant which contained the ethanoic cell extract was collected.

Antimicrobial Assays Against Streptococcus bovis, Fusobacterium Necrophorum, Escherichia coli O157 and Arcanobacterium pyogenes

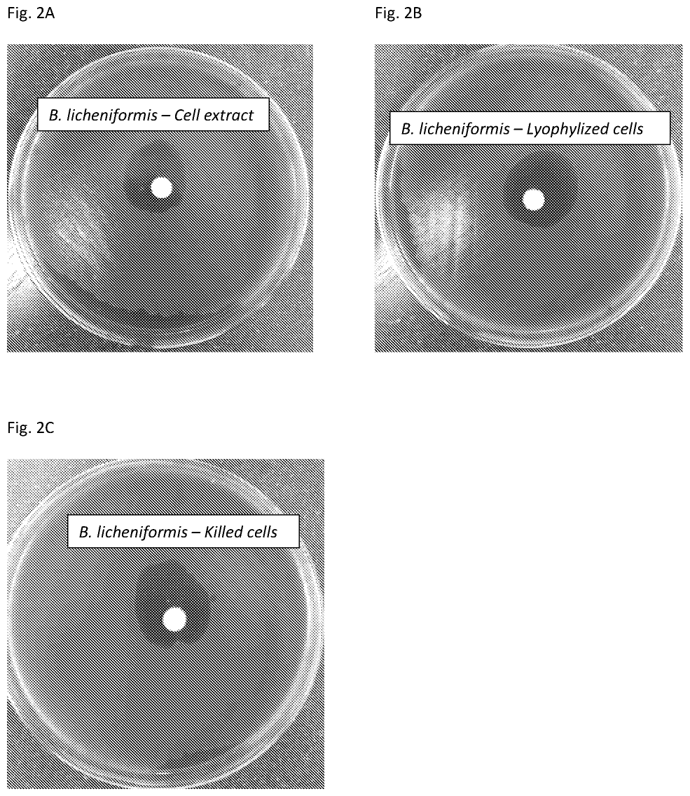

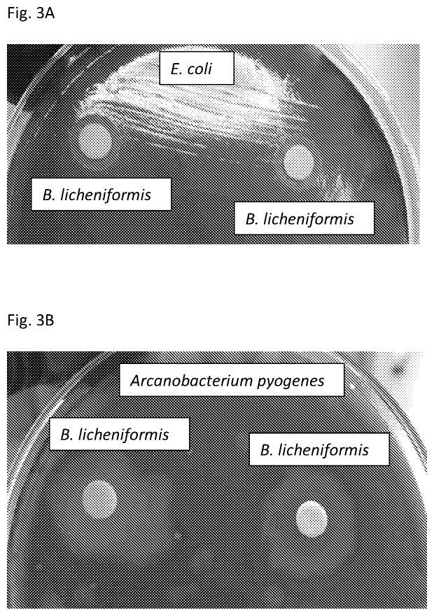

Both the ethanolic cell extract and live cells were used for agar disk diffusion antimicrobial assays. The Kirby-Bauer Disk Diffusion Susceptibility test was performed as described previously with the exception of using Mueller Hinton agar plates with 5% sheep blood (purchased from BD BBL™, Sparks, Md.) instead of regular Mueller Hinton agar plates without blood. This was done because of the fastidious nature of Fusobacterium necrophorum. All incubations for the antimicrobial assays were performed in an anaerobic jar at 37° C. within the anaerobic chamber (See, FIGS. 2A-2C). Additionally, live culture assays using Escherichia coli O157 and Arcanobacterium pyogenes was performed to evaluate the ability of B. licheniformis to inhibit growth of these pathogens (FIGS. 3A and 3B).

### Example 2

Feeding Trial Using the Composition as a Direct Fed Microbial

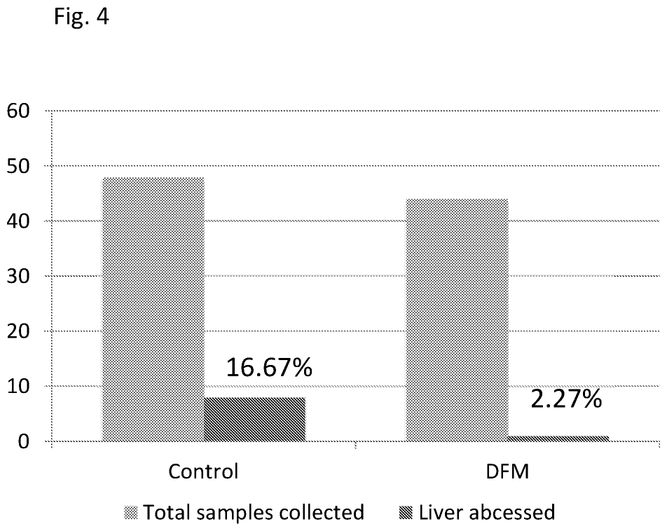

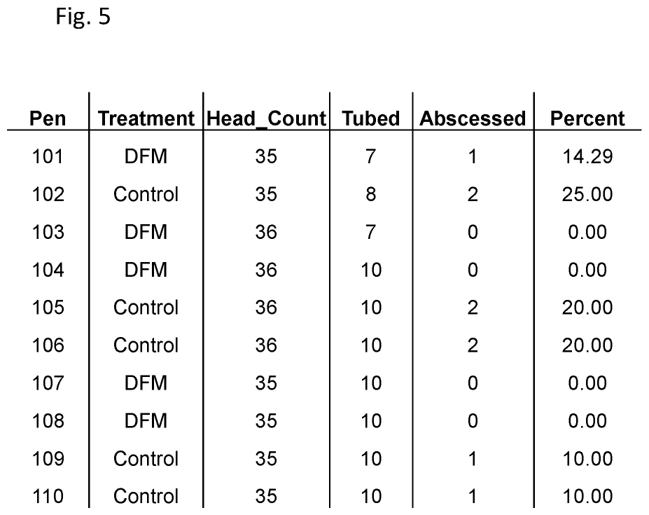

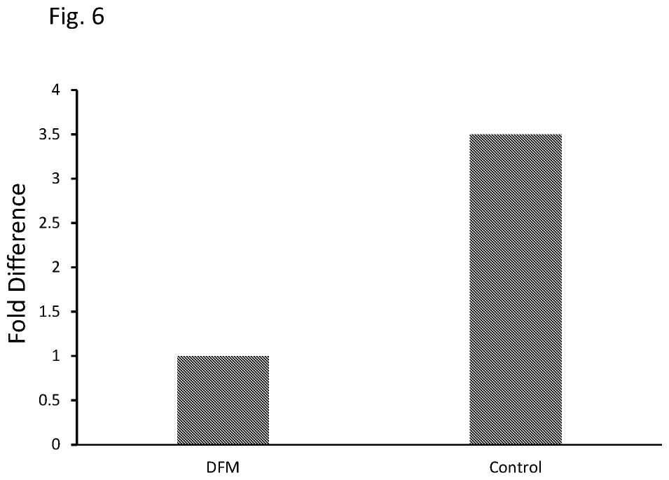

In order to assess the effectiveness of a composition in accordance with the present disclosure, a feeding trial using the composition as a direct fed microbial was performed. The diet feed consisted of 30.93% dry-rolled corn, 14.30% corn silage, 53.18% WDGS, and 1.59% of a mineral/vitamin supplement. Five groups of cattle (35-36 head of cattle in each group) were allowed to eat the diet feed and acted as controls for the study. The diet was fed for 140 day feeding period after the cattle were adapted to the finishing diet. The treatment of DFM was imposed on the base diet above where, cattle (n=150) were fed DFM at a concentration of 1 billion cells per head per day. Five other groups of cattle (35-36 head of cattle in each group) were fed the same feed diet but to which a quantity of the composition was applied to render a direct fed microbial. The study design and results are provided below in Table 2. Test subjects were examined for the presence of liver abscesses at the end of the study. The results (as shown in FIG. 4) demonstrated that 16.67% of the control animals developed liver abscesses while just 2.27% of the animals that were fed the direct fed microbial composition developed liver abscesses. FIGS. 5 and 6 further illustrate that test subjects fed the direct fed microbial experienced a significant reduction in the incidence of liver abscesses.

TABLE 2

| | | | Total | | | | Total | | | |
|---|---|---|---|---|---|---|---|---|---|---|
| Pen | Treatment | Head/pen | Count | A+ | A− | A | % | A+ | A− | A |
| 101 | DFM | 35 | 4 | 3 | 1 | 0 | 11.43 | 8.57 | 2.86 | 0 |
| 102 | Control | 35 | 4 | 2 | 2 | 0 | 11.43 | 5.71 | 5.71 | 0 |
| 103 | DFM | 36 | 6 | 5 | 1 | 0 | 16.67 | 13.89 | 2.78 | 0 |
| 104 | DFM | 36 | 6 | 5 | 1 | 0 | 16.67 | 13.89 | 2.78 | 0 |
| 105 | Control | 36 | 4 | 3 | 1 | 0 | 11.11 | 8.33 | 2.78 | 0 |
| 106 | Control | 36 | 4 | 3 | 1 | 0 | 11.11 | 8.33 | 2.78 | 0 |
| 107 | DFM | 35 | 7 | 5 | 2 | 0 | 20.00 | 14.29 | 5.71 | 0 |
| 108 | DFM | 35 | 5 | 4 | 1 | 0 | 14.29 | 11.43 | 2.86 | 0 |
| 109 | Control | 35 | 11 | 7 | 2 | 2 | 31.42 | 20.00 | 5.71 | 5.71 |
| 110 | Control | 35 | 6 | 5 | 1 | 0 | 17.14 | 14.29 | 2.86 | 0 |

### Example 3

Feeding Trial Incorporating Bacillus licheniformis and Bacillus pumilus

A feeding trial was conducted at United States Meat Animal Research Center. A cohort of n=300 cattle were fed a diet containing 71.75% dry rolled corn, 9% corn stalks, 15% wet distillers grains, 0.75% urea and 3.5% vitamin supplement with monensisn. The diet was fed for 240 day feeding period. The treatment of DFM was imposed on the base diet above where, cattle (n=150) were fed DFM at a concentration of 1 billion cells per head per day. The DFMs were fed in two stages, Bacillus pumilus was only fed for the first 140 days. After further in vitro analysis, a cocktail of Bacillus pumilus and Bacillus licheniformis was fed together, each at a concentration of 1 billion cells per head per day. Before feeding the mixture of the two DFMs, rumen samples were collected through esophageal tubing to evaluate Fusobacterium populations in both the treatment and control animals. The DFM was top dressed on the feed bunks by spraying the feed bunks after feed delivery.

### Harvesting the DFM

Harvesting of DFM occurred by growing DFM in BHI at for 48 hrs, shaking, at 37° C. for 48 hours in one-liter volumes with 48 hours. Cell counting was performed using Flow Cytometry. Invitrogen's Live/Dead Assay (Life Technologies Corporation Eugene, Oreg.) stained the cells for viewing and the protocol was followed according to manufactures' directions. Spherotech 5.14 μm beads were used as a reference while conducting the flow cytometry. (Sphero ACFP-50-5 Spherotech Inc. Lake Forest, Ill.). Twenty percent glycerol was added to the culture prior to −80° C. storage. Flow cytometry was used to calculate the cfu's present in each mL. If need, culture was diluted in order to reach a concentration of one billion cells per mL.

The results of this trial showed that the DFM provided significant benefits and protection against infection and accompanying clinical signs or symptoms of the pathogens discussed herein.

1. A composition comprising:
at least one bacterial species; and
a component selected from the group consisting of one or more additional active ingredients, excipients, dissolution agents, surfactants, antioxidants, antiseptics, preservatives, penetrants, osmoprotectants, cryoprotectants, prebiotics, and any combination thereof.

2. The composition of claim 1, wherein said bacterial species synthesize and/or secrete/excrerte bacteriocins or short peptide sequences that inhibit the growth of one or more pathogens.

3. The composition of claim 1, wherein said bacterial species is a probiotic species.

4. The composition of claim 3, wherein said probiotic species is present in the composition in a form selected from the group consisting of a live bacterial culture, an inactivated or killed bacterial culture, a lyophilized bacterial preparation, a cell culture extract, or any combination thereof.

5. The composition of claim 1, wherein said bacterial species is a Bacillus species.

6. The composition of claim 1, wherein said bacterial species is Bacillus licheniformis, alone or in combination with Bacillus pumilus.

7. The composition of claim 1, wherein said bacterial species has a 16S sequence having at least 80% sequence homology or sequence identity to any one of SEQ ID NOs. 1-3.

8. The composition of claim 1, wherein said bacterial species inhibits the growth of a pathogen.

9. The composition of claim 8, wherein said pathogen is selected from the group consisting of Streptococcus, Fusobacterium, Escherichia, and Arcanobacterium.

10. The composition of claim 9, wherein said pathogen is selected from the group consisting of Streptococcus bovis, Fusobacterium necrophorum, Escherichia coli, and Arcanobacterium pyogenes.

11. (canceled)

12. (canceled)

13. (canceled)

14. (canceled)

15. (canceled)

16. (canceled)

17. The composition of claim 1, wherein said composition is in a form capable of being administered in an administration route selected from the group consisting of oral (po), inhalation, buccal, sublingual, suppository, microbiome transplantation, topical, dermal, and any combination thereof.

18. The composition of claim 1, wherein said composition is in a form selected from the group consisting of liquids, solids, tablets, pills, capsules, solids in a liquid medium, powders, lozenges, straws, sachets, cachets, solutions, elixirs, suspensions, emulsions, solutions, syrups, sprays, aerosols, soft and hard gelatin capsules, sterile packaged powders, combined with or introduced into a food product as a direct fed microbial, ointments, dips, pastes, and any combination thereof.

19. (canceled)

20. A method of decreasing the incidence of, severity of, duration of, or frequency of clinical signs of infection from a pathogen selected from the group consisting of Streptococcus, Fusobacterium, Escherichia, and Arcanobacterium, comprising the steps of:
administering a therapeutically-effective amount of the composition of claim 1 to an animal in need thereof.

21. The method of claim 20, wherein said animal in need thereof is selected from the group consisting of a human, cow, pig, sheep, deer, goat, horse, or chicken.

22. The method of claim 20, wherein said composition is administered via an administration route selected from the group consisting of oral (po), inhalation, buccal, sublingual, suppository, microbiome transplantation, topical, dermal, and any combination thereof.

23. The method of claim 20, wherein said composition comprises a Bacillus species.

24. (canceled)

25. (canceled)

26. The method of claim 23, wherein administration of said composition comprising said Bacillus species inhibits the growth of said pathogen.

27. The method of claim 20, wherein said pathogen is selected from the group consisting of Streptococcus bovis, Fusobacterium necrophorum, Escherichia coli, and Arcanobacterium pyogenes.

28. (canceled)

29. (canceled)

30. (canceled)

31. (canceled)

32. (canceled)

33. (canceled)

34. The method of claim 20, wherein said composition is in the form of a paste, spray, ointment, or direct fed microbial.

35. The method of claim 34, wherein said composition is applied as a top dressing to animal feed.


SEQUENCE LISTING (4 sequences)

SEQ ID NO: 1 - DNA, 1396 bp, Bacillus licheniformis
agtcgagcggaccgacgggagcttgctcccttaggtcagcggcggacgggtgagtaacacgtgggtaacctgcctgtaagactgggataactccgggaaaccggggctaataccggatgcttgattgaaccgcatggttcaatcataaaaggtggcttttagctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaa

SEQ ID NO: 2 - DNA, 1264 bp, Bacillus licheniformis
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacaaggtagccgtatcggaaggtgcggctggatcacctccttt

SEQ ID NO: 3 - DNA, 855 bp, Bacillus licheniformis
tcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtacaagagtaactgcttgtaccttgacggtacctaaccagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagagaagagtggaattccacgtgtagcggtgaaatgcgtagagatgtggaggaacaccagtggcgaaggcgactctttggtctgtaactgacgctgaggcgcgaaagcgtggggagcaaacaggattagataccctggtagtccacgccgtaaacgatgagtgctaagtgttagagggtttccgccctttagtgctgcagcaaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgact

SEQ ID NO: 4 - DNA, 1135 bp, Bacillus pumilus
ggagagtttgatcctggctcaggacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagca